(19) United States
(12) Patent Application Publication
(10) Pub. No.: US 2024/0024052 A1
(43) Pub. Date: Jan. 25, 2024

(54) DISTRIBUTED SAFETY NETWORK

(71) Applicant: Covidien LP, Mansfield, MA (US)

(72) Inventors: Stefan Joerg, Muenchen (DE); Srilalitha Kumaresan, Wakefield, RI (US); Jan Tully, Muenchen (DE); Sebastian Schlingmann, Augsburg (DE)

(21) Appl. No.: 18/265,448

(22) PCT Filed: Jan. 7, 2022
(86) PCT No.: PCT/US2022/011601

§ 371 Date: Jun. 6, 2023

Related U.S. Application Data
(60) Provisional application No. 63/136,716, filed on Jan. 13, 2021.

(51) Int. Cl. A61B 34/37; A61B 34/00

(52) U.S. Cl. CPC A61B 34/37; A61B 34/74; A61B 34/77; A61B 2034/304

(57) ABSTRACT

A surgical robotic system includes a control tower or surgical console including a main computer. The system also includes a plurality of surgical robotic arms each of which includes an arm computer. The system also includes a safety network configured to distribute errors from the arm computer of at least one of the plurality of the surgical robotic arms to at least one of the arm computer of at least one other of the plurality of the surgical robotic arms or the main computer.

### BACKGROUND

### Technical Field

The present disclosure generally relates to a surgical robotic system having one or more modular arm carts each of which supports a robotic arm, and a surgical console for controlling the carts and their respective arms. Each of the components of the surgical robotic system defines a node in a distributed computing system. The present disclosure is directed to a system and method for propagating errors corresponding to various fault conditions to all nodes of the distributed computing system.

### Background of Related Art

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm. Each of the components, e.g., surgical console, robotic arm, etc., of the surgical robotic system may be controlled by one or more computing devices, which communicate with each other through a communication network.

Thus, when one of the computing devices or a component of the surgical robotic system experiences an error, such error needs to be propagated through the entire surgical robotic system and all of its components, since an error may be localized or affect other components and/or the entire surgical robotic system. Accordingly, there is a need for a distributed safety network for use with surgical robotic systems.

### SUMMARY

According to one embodiment of the present disclosure, a surgical robotic system is disclosed. The surgical robotic system includes a control tower or surgical console including a main computer, a surgical console, and a plurality of surgical robotic arms each of which includes a computer. The system also includes a safety network configured to distribute errors from the computer of at least one of the plurality of the surgical robotic arms to at least one of: another computer of at least one of the plurality of the surgical robotic arms computer; or the main computer.

Implementations of the above embodiment may include one or more of the following features. According to one aspect of the above embodiment, the safety network further includes: a master safety monitor; at least one mid-level safety monitor; and a safety group including a plurality of local safety monitors. The main computer may be configured to execute the master safety monitor. The computer of one or more of the plurality of the surgical robotic arms may be configured to execute the at least one mid-level safety monitor. Each of the local safety monitors may be configured to receive a fault condition. Each of the local safety monitors includes a local lookup table storing a local response corresponding to the fault condition. Each of the local safety monitors may be configured to propagate the fault condition to the at least one mid-level safety monitor based on the local response. The at least one mid-level safety monitor includes a mid-level lookup table storing a mid-level response corresponding to the fault condition. The at least one mid-level safety monitor may be configured to propagate the fault condition to the master safety monitor based on the mid-level response. The master safety monitor includes a master lookup table storing a master response corresponding to the fault condition. The master safety monitor may be configured to issue a master command to the at least one mid-level safety monitor based on the master response. The at least one mid-level safety monitor may be configured to issue a mid-level command to the safety group based on the mid-level response.

According to another embodiment of the present disclosure, a method for distributing errors through a surgical robotic system is disclosed. The method includes providing a fault condition of a component of a surgical robotic arm to at least one local safety monitor of a plurality of local safety monitors and propagating the fault condition to a mid-level safety monitor. The method also includes outputting a response at the mid-level safety monitor based on the fault condition including supplying a command to each of the plurality of local safety monitors.

Implementations of the above embodiment may include one or more of the following features. According to one aspect of the above embodiment, the method may further include: accessing a local lookup table stored at the local safety monitor, the local lookup table storing a local response corresponding to the fault condition. The fault condition may be propagated to the mid-level safety monitor based on the local response. The method may also include accessing a mid-level lookup table stored at the mid-level safety monitor, the mid-level lookup table storing a mid-level response corresponding to the fault condition. The method may further include propagating the fault condition to a master safety monitor from the mid-level safety monitor based on the mid-level response. The method may also include accessing a master lookup table stored at the master safety monitor, the master lookup table storing a master response corresponding to the fault condition. The method may further include issuing a master command from the master safety monitor to the mid-level safety monitor based on the master response. The method may additionally include issuing a mid-level command from the mid-level safety monitor to a safety group based on the mid-level response, the safety group including the plurality of local safety monitors.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a schematic diagram of a safety network for distributing errors through the system according to an embodiment of the present disclosure; and

FIG. 6 is a flow chart of a method for distributing errors through the system according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, a personal computer, or a server system.

As will be described in detail below, in one aspect the present disclosure is directed to a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command. In embodiments, the surgical robotic system may implement the functionality of the control tower by including a main safety monitor according to the present disclosure in the surgical console.

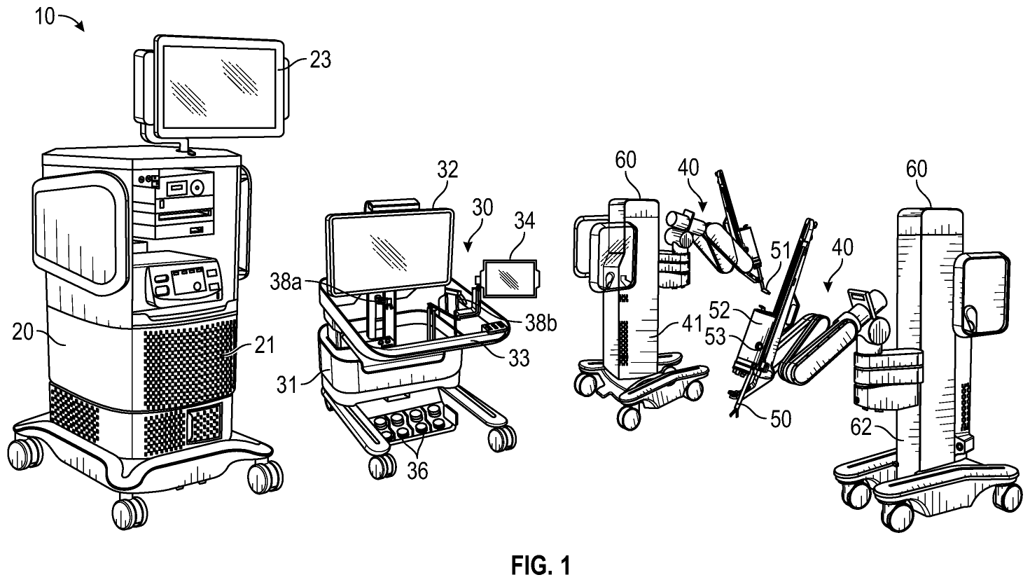

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include a camera 51 configured to capture video of the surgical site. The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of hand controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the hand controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), datagram congestion control protocol (DCCP), and/or DDS Real-Time Publish-Subscribe Protocol (RTPS). Wireless communication may be achieved via one or more wireless configurations, e.g., cellular (Edge, 3G, 4G, and 5G) and other radio frequency communications, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)). Additionally, embedded computing protocols may also be used, such as Serial Peripheral Interface (SPI) and Ethernet for Control Automation Technology (EtherCAT).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

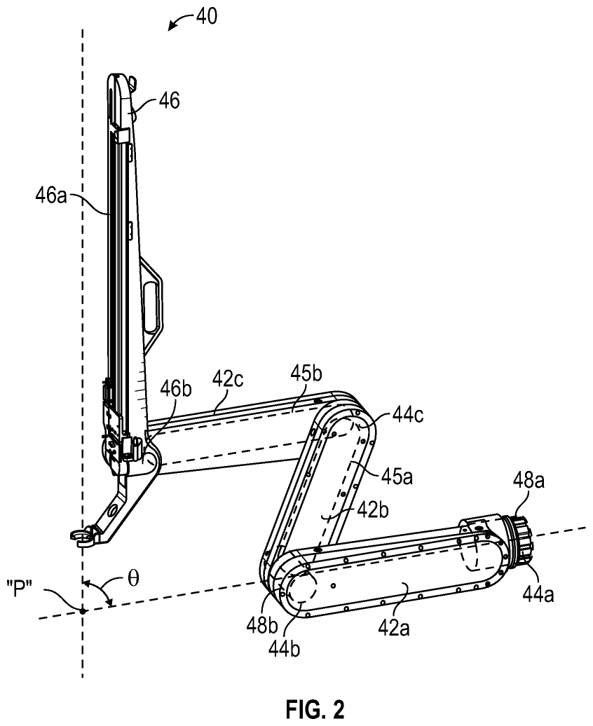

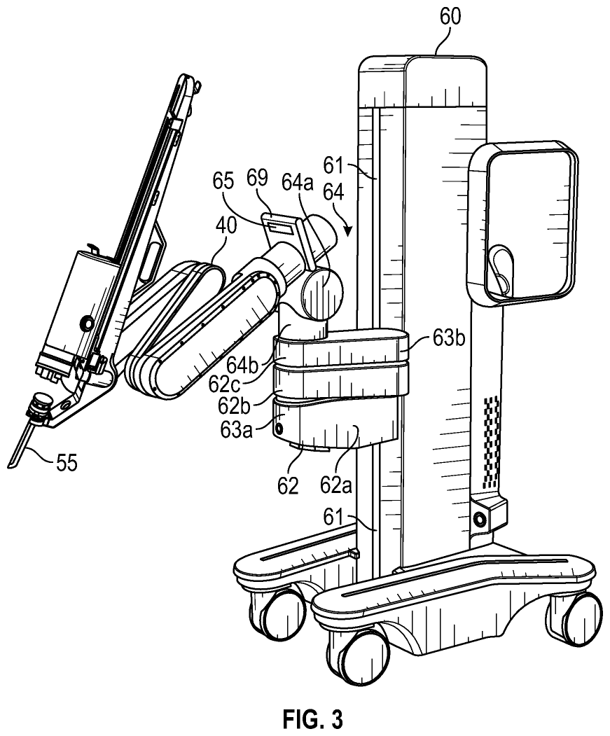

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

With reference to FIG. 3, the setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46.

The robotic arm 40 also includes a plurality of manual override buttons 53 (FIG. 1) disposed on the IDU 52 and the setup arm 62, which may be used in a manual mode. The user may press one or more of the buttons 53 to move the component associated with the button 53.

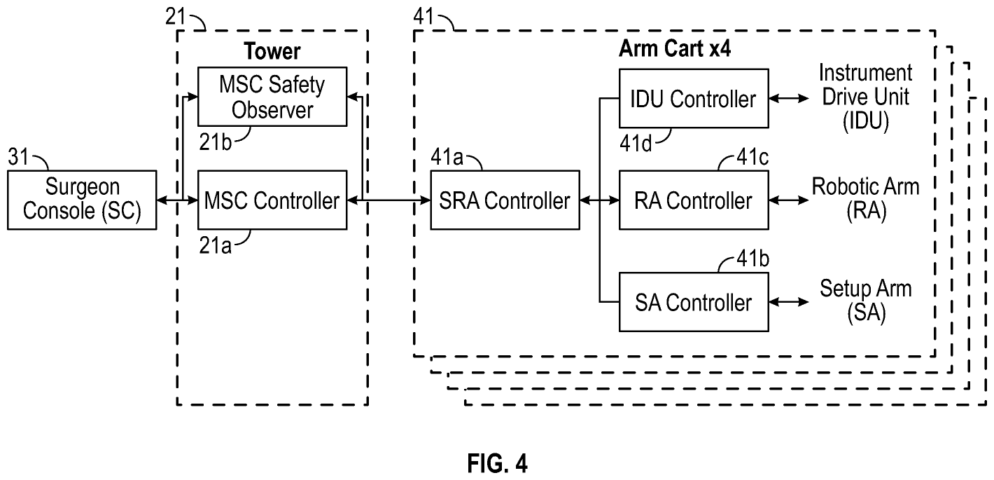

With reference to FIG. 4, each of the computers 21, 31, 41 of the surgical robotic system 10 may include a plurality of controllers, which may be embodied in hardware and/or software. The computer 21 of the control tower 20 includes a main controller 21a and safety observer 21b. The main controller 21a receives data from the computer 31 of the surgical console 30 about the current position and/or orientation of the hand controllers 38a and 38b and the state of the foot pedals 36 and other buttons. The main controller 21a processes these input positions to determine desired drive commands for each joint of the robotic arm 40 and/or the IDU 52 and communicates these to the computer 41 of the robotic arm 40. The main controller 21a also receives the actual joint angles measured by encoders of the actuators 48a and 48b and uses this information to determine force feedback commands that are transmitted back to the computer 31 of the surgical console 30 to provide haptic feedback through the hand controllers 38a and 38b. The safety observer 21b performs validity checks on the data going into and out of the main controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the main controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the main controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the main controller 21a. The hand eye function, as well as other functions described herein, is/are embodied in software executable by the main controller 21a or any other suitable controller described herein. The pose of one of the hand controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the hand controller 38a is then scaled by a scaling function executed by the main controller 21a. In embodiments, the coordinate position is scaled down and the orientation is scaled up by the scaling function. In addition, the main controller 21a also executes a clutching function, which disengages the hand controller 38a from the robotic arm 40. In particular, the main controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the main controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

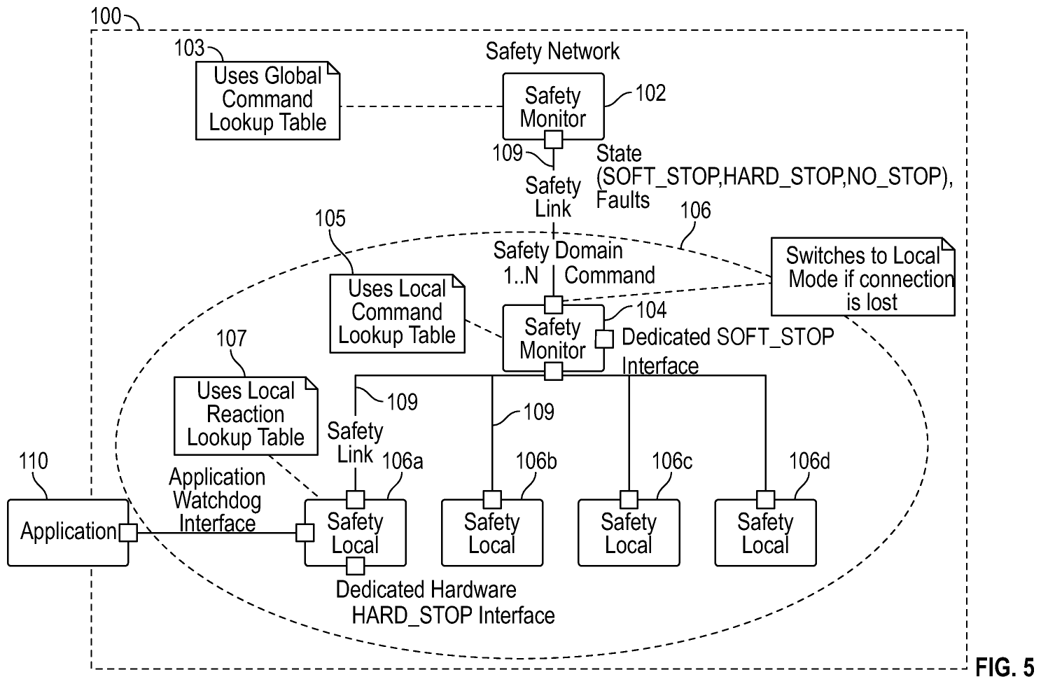

With reference to FIG. 5, a safety network 100 for distributing errors through the system 10 when fault conditions are encountered. As used herein, the term “fault condition” is any error condition encountered by any of the software applications executed by the computers 21, 31, 41 of the surgical robotic system 10. Due to the distributed architecture of the system 10, i.e., with the robotic arms 40 being separate from each other as well as the control tower 20, the computers 21, 31, 41 are networked and are configured in a distributed system. The safety network 100 is a hierarchical network of processes which propagate fault conditions in the distributed system. The safety network 100 also propagates the responses to these fault conditions to all nodes in the distributed system. Propagation of fault conditions and responses thereto allow for a coordinated response, i.e., placing the system 10 into a safe state. As used herein the term “node” denotes any component of the system 10, including the control tower 20, the surgical console 30, the robotic arm 40, movable cart 60, the setup arm 62, the instrument 50, the IDU 52, etc. Thus, the robotic arm 40 includes a plurality of computer devices connected to the distributed safety network 100 with local safety monitors running on each computer device. The setup arm 62 also includes a plurality of computer devices connected to the distributed safety network 100 with local safety monitors running on each computer device.

The safety network 100 includes multiple layers of master and slave processes. The top layer includes a master safety monitor 102 that receives the “controller mode” from the main controller 21a and shares its state (hardware stop [hard_stop], software stop [soft_stop], no_stop) with the main controller 21a. The master safety monitors 102 coordinates one or more mid-level safety monitors 104 (1 . . . N) and local safety monitors 106a-d to form a coordinated response to a fault condition on one or more nodes in the system. The local safety monitors 106a-d are arranged in a safety group 106. The mid-level safety monitor 104 gets the “controller mode” from main cart controller 41a and shares its state (hard_stop, soft_stop, no_stop) with the main cart controller 41a. Each of the local safety monitors 106a-d monitors the main cart controller 41a, the setup arm controller 41b, the robotic arm controller 41c, and the IDU controller 41d and receives input from the applications 110 being run on these controllers. Thus, the mid-level safety monitor 104 is a slave (e.g., follower) to the master safety monitor 102 and a master (e.g., leader) to the local safety monitors 106a. The master safety monitor 102, the mid-level safety monitor 104, and the local safety monitors 106a-d communicate with each other through safety links 109.

Software applications 110 may output fault conditions during their execution by the computers 21, 31, 41. Such software applications include all applications that are outside the safety network 100, e.g., those directed to movement control, camera control, etc. The software applications 110 detect and report fault conditions to their respective local safety monitors 106a-d. In the event of a local fault condition, each of the local safety monitors 106a-d places the corresponding node in a safe state, i.e., a state in which the node is powered but does not execute any other software applications aside from the local safety monitors 106a-d. Each of the local safety monitors 106a-d has their own local lookup table 107, which are used to determine the local response. The local lookup table 107 stores a list of fault conditions, and for each fault condition, the lookup table 107 stores a corresponding response, which includes entering a safe state or shutting down of a local node and/or propagating the fault condition to the mid-level safety monitor 104.

Each fault condition is also propagated to the corresponding mid-level safety monitor 104. The mid-level safety monitors 104 have mid-level lookup tables 105 used to command a safe state to the safety group 106 of nodes based on a fault condition. In particular, the mid-level lookup table 105 stores a list of fault conditions, and for each fault condition, the mid-level lookup table 105 stores the safety group 106 and/or a list of nodes and corresponding responses, e.g., commands. Thus, upon receiving the fault condition, the mid-level safety monitor 104 looks up the list of nodes and corresponding responses and instructs the nodes to execute the commands based on the fault condition. Suitable commands include hard_stop, soft_stop (only between main and mid-level), and hard_stop. Therefore, mid-level safety monitor 104 has an interface to trigger a dedicated soft_stop action that is specific to the safety group 106. A local safety monitor 106a-d may have a means/interface for a dedicated hard_stop action that is specific to the local safety monitor 106a-d.

Faults are propagated up from local safety monitors 106a-d to mid-level safety monitor 104 and, if needed, to the master safety monitor 102. The master safety monitor 102 also includes a global lookup table 103, which stores a list of fault conditions, and for each fault condition, the lookup table 103 stores a list of nodes or safety groups and corresponding responses, e.g., commands for each of the safety groups. Similarly, commands are propagated down from the master safety monitor 102 to the mid-level safety monitor 104 and subsequently to the local safety monitors 106a-d. The master safety monitor 102 determines which commands and nodes that are affected by the fault condition from the lookup table 103.

The safety links 109 provide reliable transport and may include a plurality of data verification checks, such as cyclic redundancy checks, timing checks, and flow control. These checks allow the ability to check for message delay, corruption, repetition, loss and incorrect addressing. In the event of a transmission error, the safety link 109 is be reset. If the connection between master safety monitor 102 and the mid-level safety monitor 104, i.e., the safety link 109, is lost, the mid-level safety monitor 104 can independently act in a local mode, thereby propagating commands and fault conditions to the local safety monitors 106a-d.

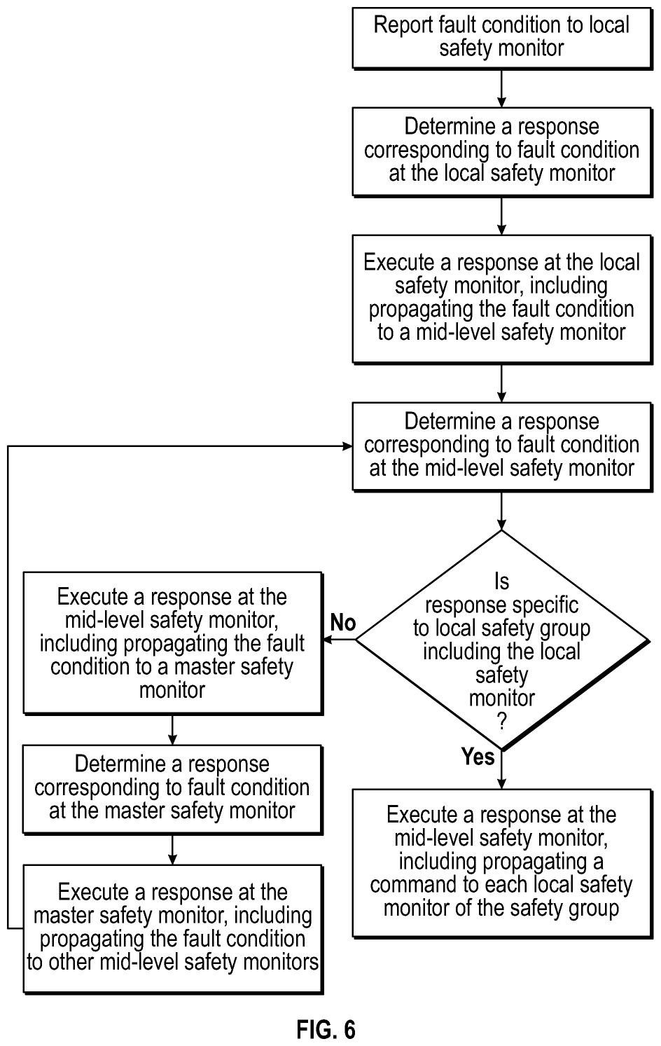

With reference to FIG. 6, a method for distributing errors through the safety network 100 is disclosed. Initially, a fault condition is output by the software application 110 being executed by any of the computers 21, 31, 41 and is provided to one of the local safety monitors 106a-d. The corresponding local safety monitor 106a-d, e.g., 106a, accesses the local lookup table 107 to retrieve an action or a series of actions corresponding to the fault condition. The action may include instructing the corresponding node to enter the safe state and/or the hardware stop state. The action may also include propagating the fault condition to the mid-level safety monitor 104.

The mid-level safety monitor 104 accesses its mid-level lookup table 105 to determine an action or a series of actions corresponding to the fault condition propagated from one of the local safety monitors 106a-d. Initially the mid-level safety monitor 104 determines whether the fault condition applies only the safety group 106 or to other nodes that are part of other safety groups. If the fault condition is specific only to the safety group 106, the mid-level safety monitor 104 commands the rest of the safety monitors 106b-d, to take appropriate action, which may be the same as those taken by the safety monitor 106a. In embodiments, the actions taken by the safety monitors 106b-d may be different from the actions taken by the safety monitor 106a.

If the fault condition is determined to be applicable to nodes outside the safety group 106, the mid-level safety monitor 104 propagates the fault condition to the master safety monitor 102. In addition to propagating the fault condition, the mid-level safety monitor 104 also propagates commands through the safety group 106. Upon receiving the fault condition, the master safety monitor 102 accesses the global lookup table 103, to determine which nodes and safety groups are affected by the fault condition. The master safety monitor 102 then propagates a command to each of the affected safety groups 106 and their respective mid-level safety monitors 104, which then take appropriate action and propagate the command to their respective local safety monitors 106a-d. The hierarchal distributive system allows for propagation of fault conditions and commands in a focused manner, allowing unaffected nodes and their respective safety groups to continue operation in situations where faults conditions are localized and do not widely affect operation of the system 10.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system including:
a control tower or a surgical console including a main computer;
a plurality of surgical robotic arms each of which includes an arm computer; and
a safety network configured to distribute errors from the arm computer of at least one of the plurality of the surgical robotic arms to at least one of the arm computer of at least one other of the plurality of the surgical robotic arms or the main computer.

2. The surgical robotic system according to claim 1, wherein the safety network includes:
a master safety monitor;
at least one mid-level safety monitor; and
at least one safety group including a plurality of local safety monitors.

3. The surgical robotic system according to claim 2, wherein the main computer is configured to execute the master safety monitor.

4. The surgical robotic system according to claim 2, wherein the arm computer of at least one of the plurality of the surgical robotic arms is configured to execute the at least one mid-level safety monitor.

5. The surgical robotic system according to claim 2, wherein each of the local safety monitors is configured to receive a fault condition.

6. The surgical robotic system according to claim 5, wherein each of the local safety monitors includes a local lookup table storing a local response corresponding to the fault condition.

7. The surgical robotic system according to claim 6, wherein each of the local safety monitors is configured to propagate the fault condition to the at least one mid-level safety monitor based on the local response.

8. The surgical robotic system according to claim 7, wherein the at least one mid-level safety monitor includes a mid-level lookup table storing a mid-level response corresponding to the fault condition.

9. The surgical robotic system according to claim 8, wherein the at least one mid-level safety monitor is configured to propagate the fault condition to the master safety monitor based on the mid-level response.

10. The surgical robotic system according to claim 8, wherein the at least one mid-level safety monitor is configured to issue a mid-level command to the at least one safety group based on the mid-level response.

11. The surgical robotic system according to claim 9, wherein the master safety monitor includes a master lookup table storing a master response corresponding to the fault condition.

12. The surgical robotic system according to claim 11, wherein the master safety monitor is configured to issue a master command to the at least one mid-level safety monitor based on the master response.

13. A method for distributing errors through a surgical robotic system, the method comprising:
providing a fault condition of a component of a surgical robotic arm to at least one local safety monitor of a plurality of local safety monitors;
propagating the fault condition to a mid-level safety monitor; and
outputting a response at the mid-level safety monitor based on the fault condition including supplying a command to each of the plurality of local safety monitors.

14. The method according to claim 13, further comprising:
accessing a local lookup table stored at the local safety monitor, the local lookup table storing a local response corresponding to the fault condition.

15. The method according to claim 14, wherein the fault condition is propagated to the mid-level safety monitor based on the local response.

16. The method according to claim 15, further comprising:
accessing a mid-level lookup table stored at the mid-level safety monitor, the mid-level lookup table storing a mid-level response corresponding to the fault condition.

17. The method according to claim 16, further comprising:
propagating the fault condition to a master safety monitor from the mid-level safety monitor based on the mid-level response.

18. The method according to claim 16, further comprising:
issuing a mid-level command from the mid-level safety monitor to at least one safety group based on the mid-level response, the at least one safety group including the plurality of local safety monitors.

19. The method according to claim 17, further comprising:
accessing a master lookup table stored at the master safety monitor, the master lookup table storing a master response corresponding to the fault condition.

20. The method according to claim 19, further comprising:
issuing a master command from the master safety monitor to the mid-level safety monitor based on the master response.
